(19) United States
(12) Patent Application Publication
(10) Pub. No.: US 2019/0002585 A1
(43) Pub. Date: Jan. 3, 2019

(54) CANCER TREATMENT METHODS AND COMPOSITIONS

(71) Applicant: The Cleveland Clinic Foundation, Cleveland, OH (US)

(72) Inventors: Robert H. Silverman, Cleveland, OH (US); Shuvojit Banerjee, Cleveland, OH (US)

(21) Appl. No.: 16/002,295

(22) Filed: Jun. 7, 2018

Related U.S. Application Data
(60) Provisional application No. 62/516,909, filed on Jun. 8, 2017.

(51) Int. Cl. C07K 16/40; A61P 35/02; C12N 15/113; A61P 35/04; A61K 31/7068; A61K 31/7076

(52) U.S. Cl. CPC C07K 16/40; A61P 35/02; A61K 31/7076; A61P 35/04; A61K 31/7068; C12N 15/1137

(57) ABSTRACT

Provided herein are compositions, systems, kits, and methods for treating cancer by administering to a subject a first agent selected from: a PDE12 inhibiting agent, a AKAP7 inhibiting agent, an ADAR1 inhibiting agent, and/or a RNase L enhancer, and a second agent that comprises a DNA methylation inhibiting agent (e.g., a DNA methyltransferase demethylase inhibitor).

The present application claims priority to U.S. provisional application Ser. No. 62/516,909, filed Jun. 8, 2017, which is herein incorporated by reference in its entirety.

### STATEMENT REGARDING FEDERAL FUNDING

This invention was made with government support under grants nos. CA044059 and AI104887 awarded by the National Institutes of Health. The government has certain rights in the invention.

### FIELD

Provided herein are compositions, systems, kits, and methods for treating cancer by administering to a subject a first agent selected from: a PDE12 inhibiting agent, a AKAP7 inhibiting agent, an ADAR1 inhibiting agent, and/or a RNase L enhancer, and a second agent that comprises a DNA methylation inhibiting agent (e.g., a DNA methyltransferase demethylase inhibitor).

### BACKGROUND

Cancer is the name given to a collection of related diseases. In all types of cancer, some of the body's cells begin to divide without stopping and spread into surrounding tissues. Cancer can start almost anywhere in the human body, which is made up of trillions of cells. Normally, human cells grow and divide to form new cells as the body needs them. When cells grow old or become damaged, they die, and new cells take their place. When cancer develops, however, this orderly process breaks down. As cells become more and more abnormal, old or damaged cells survive when they should die, and new cells form when they are not needed. These extra cells can divide without stopping and may form growths called tumors. Many cancers form solid tumors, which are masses of tissue. Cancers of the blood, such as leukemias, generally do not form solid tumors. Cancerous tumors are malignant, which means they can spread into, or invade, nearby tissues. In addition, as these tumors grow, some cancer cells can break off and travel to distant places in the body through the blood or the lymph system and form new tumors far from the original tumor.

### SUMMARY

Provided herein are compositions, systems, kits, and methods for treating cancer by administering to a subject a first agent selected from: a PDE12 inhibiting agent, a AKAP7 inhibiting agent, an ADAR1 inhibiting agent, and/or a RNase L enhancer, and a second agent that comprises a DNA methylation inhibiting agent (e.g., a DNA methyltransferase demethylase inhibitor).

In some embodiments, provided herein are methods of treating cancer comprising: administering to a subject with cancer a first agent and a second agent, wherein the first agent comprises a PDE12 inhibiting agent, a AKAP7 inhibiting agent, an ADAR1 inhibiting agent, and/or a RNase L enhancer, and wherein the second agent comprises a DNA methylation inhibiting agent. In particular embodiments, the methods further comprise treating the subject with ionizing radiation (e.g., prior to or during the administering of the first and second agents).

In certain embodiments, the cancer comprises a solid malignant cancer (e.g., breast cancer, prostate, lung, colon cancer, pancreatic cancer, liver cancer, melanoma, etc.). Examples of solid tumors cancer include, for example, sarcomas, carcinomas, and lymphomas. In some embodiments, the cancer is leukemia or a myelodysplastic syndrome cancer. In particular embodiments, the administering reduces the level of cancer in the subject. In further embodiments, the administering the first and second agents reduces the level of cancer in the subject by a larger amount than administering just the second agent. In particular embodiments, the subject is a human or a domestic mammal (e.g., horse, dog, cat, cow, pig, etc.).

In some embodiments, provided herein are systems, kits, and compositions comprising: a) a first agent comprising a PDE12 inhibiting agent, a AKAP7 inhibiting agent, an ADAR1 inhibiting agent, and/or a RNase L enhancer, and b) a second agent comprising a DNA methylation inhibiting agent.

In certain embodiments, the first and second agent are present in the same composition. In further embodiments, the first agent is present in a first container, and the second agent is present in a second container. In other embodiments, the first and second containers are present in the same packaging (e.g., box). In particular embodiments, the systems and kits further comprise: c) an agent delivery component (e.g., a hypodermic needle). In certain embodiments, the kits and systems further comprise: c) a plurality of cancer cells. In some embodiments, the plurality of cancer cells are solid malignant cancer cells.

In certain embodiments, the DNA methylation inhibiting agent comprises a DNA methyltransferase demethylase inhibitor. In particular embodiments, the DNA methylation inhibiting agent comprises 5-azacytidine (AZA) or derivative thereof. In further embodiments, the DNA methylation inhibiting agent comprises 5-aza-2′-deoxycytidine (DAC) or derivative thereof.

In some embodiments, the first agent and the second agent are part of the same composition. In other embodiments, the first agent is in a first composition and the second agent is in a second composition. In other embodiments, the RNase L enhancer comprises the 2-5A RNase L activator compound or derivatives thereof or alternative small molecule activators of RNase L. In particular embodiments, the PDE12 inhibiting agent is selected from the group consisting of: an anti-PDE12 siRNA sequence, an anti-PDE12 antisense sequence, polyclonal anti-PDE12 antibodies, a small molecule inhibitor of PDE12, and an anti-PDE12 monoclonal antibody or antigen binding fragment thereof. In other embodiments, the PDE12 inhibiting agent is selected from the group consisting of: Compounds 1-4 shown in FIG. 13, or compound A-74528a FIG. 14. In some embodiments, the AKAP7 inhibiting agent is selected from the group consisting of: an anti-AKAP7 siRNA sequence, an anti-AKAP7 antisense sequence, anti-AKAP7 polyclonal antibodies, a small molecule inhibitor of AKAP7, or an anti-AKAP7 monoclonal antibody or antigen binding fragment thereof. In certain embodiments, the ADAR1 inhibiting agent comprises 8-azaadenosine (8-aza), or an antibody that binding human ADAR1 (e.g., ADAR1 Antibody (15.8.6): sc-73408, from Santa Cruz Biotechnology) or antigen binding portion thereof.

### DESCRIPTION OF THE FIGURES

FIG. 1 shows a hypothetical mechanism for cancer therapy and for how DNA methyltransferase inhibitors (DNMTi's), 5-azacytidine (AZA) and 5-aza-2′-deoxycytidine (DAC) induce formation of dsRNA in cancer cells. The dsRNA activates OAS enzymes that synthesize the RNase L activator 2-5A from ATP. RNA degradation by RNase L can lead to apoptosis of the cancer cells. The phosphodiesterase PDE12 degrades 2-5A and thus limits the activity of RNase L. Therefore, inhibition or ablation of PDE12 enhances the death of cancer cells in response to AZA or DAC or similar agents.

FIG. 2 shows the chemical structures of cytosine, 5-azacytidine (5-AZA-cyt) and 5-aza-2′-deoxycytidine (5-AZA-2′-dcyt).

FIG. 3 shows RNase L is required for the death of A549 cells in response to AZA treatment. (A) WT and (B) RNase L KO A549 cells were treated with different doses of AZA (as indicated) and cell death was monitored in an IncuCyte real-time imaging system. (C) Light microscopy images of the cell cultures at 0 and 48 h post treatment with AZA.

FIG. 4 shows MAVS contributes to AZA sensitivity, but is not required for tumor cell death in response to AZA. (A,B) WT and MAVS KO A549 cell lines and (C) MAVS-RNase L double knockout (DKO) cells treated with different doses of AZA were monitored for cell survival with an IncuCyte system.

FIG. 5 shows AZA treatment of A549 cells leads to activation of apoptosis mediators, caspase3/7 in an RNase L-dependent manner. (A) WT, (B) RNase L KO, (C) MAVS KO and (D) MAVS-RNase L DKO A549 cells were treated with 50 uM AZA in the absence or presence of 50 uM Z-VAD (a pan-caspase inhibitor). Caspase3/7 activity was monitored with caspase 3/7 reagent (IncuCyte) in an IncuCyte real-time cell imaging system.

FIG. 6 shows WT and RNase L KO A549 cells were treated with 50 uM AZA for 48 hrs. PARP cleavage was monitored in Western blots probed with [anti PAPR1 (rabbit polyclonal) (Santa Cruz Biotechnology)]. β-actin levels were monitored with 3-actin (mouse monoclonal) antibody (Sigma-Aldrich) to evaluated as a control.

FIG. 7 shows relative AZA sensitivity of (A) WT vector control A549 lung adenocarcinoma cells, (B) human primary alveolar epithelial cells, HAEC, ex vivo 13), (C) human bronchial epithelium BEAS 2B cells, and (D) human bronchial epithelial cells BETA as determined with an IncuCyte live-cell imaging system.

FIG. 8 shows sensitivity of (A,B) DU145 and (C,D) C4-2 human prostate cancer cells to AZA treatment as determine in an IncuCyte real-time cell imaging system.

FIG. 9: (A) PC3-WT and (B) PC3-RNase L-KO human prostate cancer cells untreated or treated with AZA (12.5 uM) for 48 hrs visualized with an IncuCyte real-time cell imaging system. (C) AZA sensitivity of PC3-WT and PC3-RNase L-KO cells.

FIG. 10 shows compound 2-5A greatly enhances the tumor cell-lethality of AZA. (A,C) WT or (B,D) RNase L KO A549 cells were incubated in the absence or presence of 25 uM AZA and either mock transfected with lipofectamine (Lipo) or transfected with 1 uM of 2-5A as indicated. Cell survival was determined with an IncuCyte cell imaging system.

FIG. 11 shows A549 cell survival during treatment with 50 uM AZA compared to WT, AKAP7 KO, PDE12 KO, and AKAP7-PDE12 DKO cells. Mock refers to untreated cells (i.e., WT, AKAP7 KO, PDE12 KO, AKAP-PDE12 DKO cells without AZA).

FIG. 12 shows 5-aza-2′-deoxycytidine (DAC) causes death of A549 cells through an RNase L-dependent mechanism.

FIG. 13 shows small molecule PDE12 inhibitors Compounds 1-4 from Wood et al., J Biol Chem. 2015. 290, 19681-19696.

FIG. 14 shows the small molecule inhibitor of PDE12 A-74528a from Kubota K, et al. J Biol Chem. 2004; 279(36):37832-37841.

FIG. 15 shows the molecular structure of the 2-5A compound (see, Kerr and Brown, PNAS, 1978 January; 75(1):256-60).

FIG. 16 panel (A) shows a Western blot from RNASEL KO A549 cells transduced with lentivirus expressing either wild type (WT) human RNase L or a catalytically inactive mutant human RNase L (R667A). Both WT and R667A RNase L have a FLAG epitope attached. Cells were lysed after 72 hr and proteins were analyzed by Western blot probed with anti-Flag M2 antibody (Sigma-Aldrich) (upper) and anti-β-actin antibody (Sigma-Aldrich) (lower). In panel (B) RNASEL KO cells were transduced with lentivirus expressing either WT RNase L or catalytic inactive mutant (R667A) RNase L and then treated with 50 μM of AZA for 72 hr. Dead cells and total cells were determined with an IncuCyte real-time imaging system using a dual-dye method. A minimum of three wells were treated with each experimental condition. The data are average±SD from three independent replicates. K-in, knock-in of either WT or R667A RNase L. Panel (C) shows WT human mammary epithelial cells (HME) and OAS (1, 2, 3) triple KO (TKOs) HME cells grown in 24 well and treated with different concentration of freshly prepared AZA (254M or 50 μM) and assessed for dead cells and total cells using an IncuCyte system. Three separate wells were treated with each experimental condition and a minimum of 4 image fields (>10,000 cells/well) were analyzed per well.

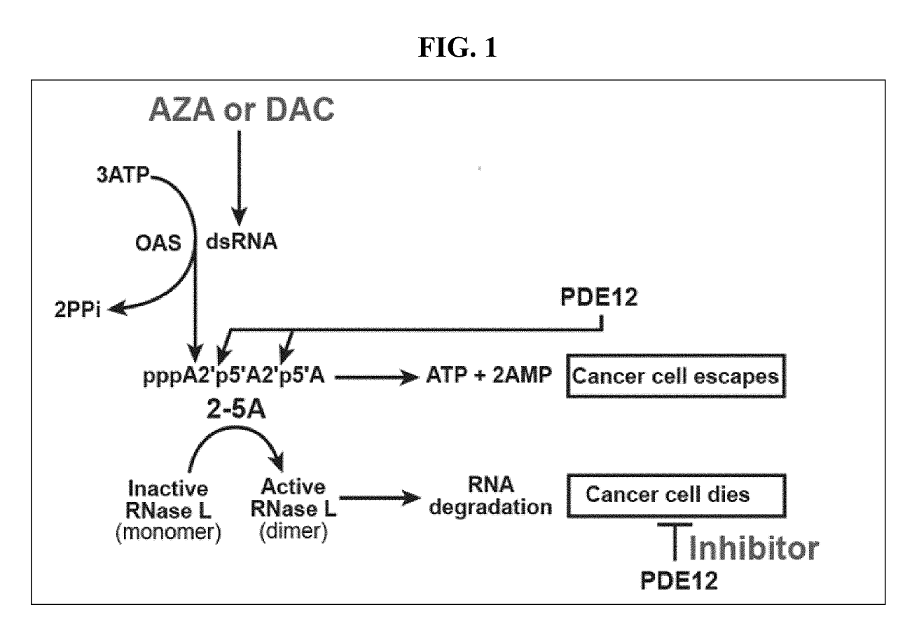

In FIG. 17 WT and RNase L KO A549 cells were grown in 96 well plates. Cells were pre-treated with JNK-Inhibitor SP600125 (25 μM) (Santa Cruz Biotechnology, Cat number # sc-200635) for 2 hr and then incubated without or with AZA (25 or 50 μM) for 60 hrs. Percent cell survival after AZA treatment was calculated using an IncuCyte system. The data are average±SD from six independent replicates. Similar data were obtained from two additional independent experiments.

In FIG. 18 WT and RNase L KO A549 cells were grown in 24 well plates. The cells were treated with or without ionizing radiation (IR) (10 Gy) for 10 min and then incubated without AZA (panel A) or with AZA at 25 μM (panel B) or 12.5 μM (panel C) for 50 hr Percent cell survival was calculated using an IncuCyte system. The data are average±SD from three independent replicates.

In FIG. 19 WT, RNase L-ADAR1 DKO, MAVS-ADAR1 DKO, MAVS-ADAR1-RNase L TKO and p150 ADAR1 isoform KO A549 cells were incubated without AZA (panel A) or with 50 mM AZA (panels B and C). Percent survival after 50LM AZA treatment was calculated using an IncuCyte system. The data are average=±SD from three independent replicates. Similar data were obtained from two additional independent experiments.

### DETAILED DESCRIPTION

Provided herein are compositions, systems, kits, and methods for treating cancer by administering to a subject a first agent selected from: a PDE12 inhibiting agent, a AKAP7 inhibiting agent, an ADAR1 inhibiting agent, and/or a RNase L enhancer, and a second agent that comprises a DNA methylation inhibiting agent (e.g., a DNA methyltransferase demethylase inhibitor).

Agents that reverse epigenetic silencing, such as the DNA methyltransferase demethylase inhibitors (DNMTi) 5-azacytidine (AZA) and 5-aza-2′-deoxycytidine (DAC), have profound effects on transcription and on tumor cell survival. AZA is an FDA-approved drug for myelodysplastic syndromes that has also be used to treat acute myeloid leukemia, and both AZA and DAC are under investigation for use against a wide range of different solid malignant tumors. Prior reports show that AZA and DAC treatments generate self-dsRNA transcribed from hypomethylated repetitive elements, including endogenous retrovirus (ERV) genes. The self-dsRNA accumulation in AZA/DAC treated cells leads to induction of type I interferon (IFN), expression of IFN stimulated genes (ISGs), an antiviral response and cell death by apoptosis. However, the specific ISGs responsible for the apoptotic activity of AZA and DAC are unknown.

In the present disclosure, it is reported that selective death of tumor cells by AZA or DAC treatment is mediated by the IFN inducible and dsRNA-dependent OAS-RNase L pathway. OASs1-3 are IFN induced enzymes that synthesize the RNase L activator 2-5A from ATP in response to dsRNA. Work conducted during development of embodiments of the present disclosure found that AZA treatment resulted in the RNase L-dependent elimination of lung or prostate tumor cells, whereas normal cell types were resistant. Similarly, gene knockouts of 2′-phosphodiesterases (PDE12 and AKAP7) that degrade 2-5A enhanced the tumor cytotoxic effects of AZA, presumably by prolonging the half-life of 2-5A and thus the activation of RNase L in tumor cells. These finding indicate that activation of RNase L in tumor cells contributes to the antitumor activity of DNA demethylating drugs. We also show that preventing or inhibiting PDE12 activity when used in combination with a DNA demethylation inhibitor, in particular AZA, will cause targeted death of cancer cells. In addition, we show that 2-5A enhances the tumor cell-lethality of AZA treatment.

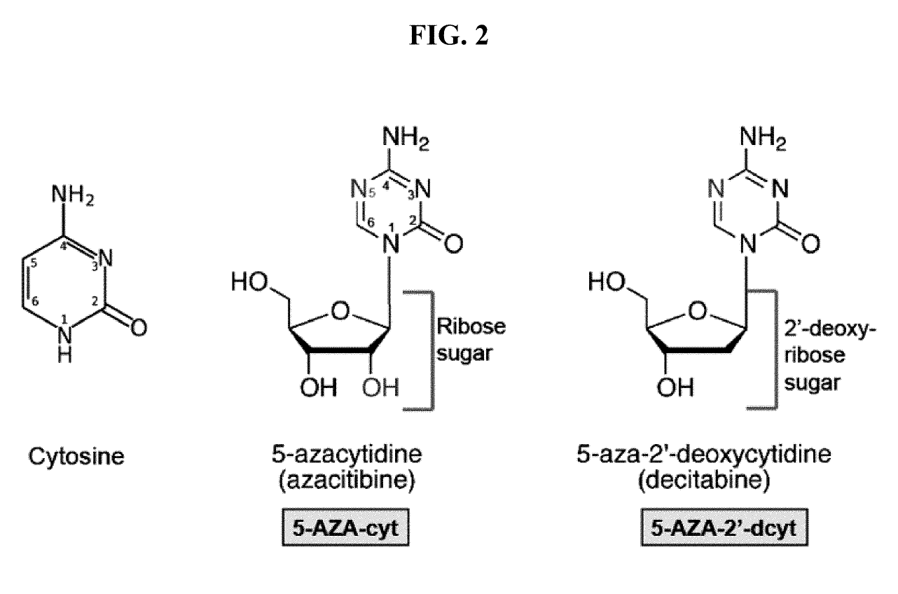

The DNA methyltransferase inhibitor (DNMTi) 5-azacytidine (AZA) is used for the treatment of myelodysplasic syndromes (MDS) and hematologic malignancy (acute myeloid leukemia)[2-5]. AZA and 5-aza-2′-deoxycytidine (DAC) are also being investigated for the treatment of solid malignant tumors (chemical structures of AZA and DAC are shown in FIG. 2). Methylation of cytosines at CpG dinucleotide islands in promoter elements results in epigenetic silencing of transcription initiation. By causing DNA hypomethylation DNMTi's have wide-ranging effects on cancer, including inducing transcription of some tumor suppressor genes [6]. Furthermore, DNA methylation and the tumor suppressor p53 cooperate to suppress transcription of repetitive DNA elements, including short interspersed repeats (SINE), satellite repeats, endogenous IAP retroviral DNA, and ncRNA genes [7]. 5-aza-2′-deoxycytidine (DAC) treatment of cells with mutant p53 led to double-stranded RNA (dsRNA) accumulation originating at least in part from transcription of repetitive DNA elements and a suicidal type I IFN response (referred to as “TRAIN”, TRanscription of Repeats Activates INterferon) [7]. DsRNA is perhaps the most common viral pathogen associated molecular pattern (PAMP), producing viral mimicry in DNMTi-treated tumor cells characterized by production of type I IFNs, transcription of IFN stimulated genes (ISGs) and an innate immune response that sometimes culminates in apoptosis. Similarly, DNA demethylation by DNMTi treatments was shown to induce the bi-directional transcription of human endogenous retrovirus-like genes (ERVs) lead to formation of dsRNA and viral mimicry [8, 9]. However, the connection between self-dsRNA, viral mimicry and tumor cell death by apoptosis are far less clear.

The 2′,5′-oligoadenylate synthetase (OAS)-RNase L pathway is an IFN stimulated antiviral response that requires dsRNA [10, 11]. Type I and type III IFNs stimulate transcription of a family of human OAS genes for enzymatically active OAS1-3 isozymes and enzymatically-inactive OASL. DsRNA binds to and activates OASs1-3 which then use ATP as substrate to synthesize a series of 5′-triphosphorylated, 2′,5′-linked oligoadenylates (2-5A). The only well-established function of 2-5A is activation of RNase L [12]. 2-5A binds to the inactive and latent monomeric form of RNase L inducing dimerization and activation (also requiring ATP or ADP binding to the protein kinase-like domain of RNase L) [13, 14]. RNase L activation cleaves viral and cellular single-stranded RNA (in particular rRNA) suppressing viral replication and causing apoptosis [15, 16].

In the Example below, we report that selective tumor cell death triggered by AZA or DAC is almost entirely mediated through the OAS-RNase L pathway. Furthermore, the anti-tumor cell activity of AZA was enhanced by inactivating mutations of the host 2′-phosphodiesterases PDE12 and AKAP7. These findings indicate that combined treatment with AZA or DAC (or other DNA methylation inhibitor) with host PDE inhibitors will produce a potent and selective anti-tumor activity against, for example, solid malignant cancers, and against myelodysplastic syndromes and leukemias.

### EXAMPLES

### Example 1

This Example describes that selective death of tumor cells by AZA or DAC treatment is mediated by the IFN inducible and dsRNA-dependent OAS-RNase L pathway. OASs1-3 are IFN induced enzymes that synthesize the RNase L activator 2-5A from ATP in response to dsRNA. We report that AZA treatment resulted in the RNase L-dependent elimination of lung or prostate tumor cells, whereas normal cell types were resistant. Similarly, gene knockouts of 2′-phosphodiesterases (PDE12 (phosphodiesterase 12) and AKAP7) that degrade 2-5A enhanced the tumor cytotoxic effects of AZA, presumably by prolonging the half-life of 2-5A and thus the activation of RNase L in tumor cells. Our findings indicate that activation of RNase L in tumor cells contributes to the antitumor activity of DNA demethylating drugs. We also show that preventing or inhibiting PDE12 activity when used in combination with a DNA demethylation inhibitor, in particular AZA, will cause targeted death of cancer cells. In addition, we show that 2-5A enhances the tumor cell-lethality of AZA treatment.

### Materials and Methods

### Cell Culture.

Cell lines and viruses. Human A549 cells (ATCC® CCL-185™) validated by STR analysis were cultured in RPMI 1640 medium (Gibco) supplemented with 10% fetal bovine serum (FBS), 100U/ml penicillin and 100 μg/ml streptomycin. PC3, DU145 and C4-2 cells were grown in RPMI with 10% FBS. HAEC, BEAS 2B and BETA cells were grown in LHC-8 (Biofluids/Biosource, catalog #P141-500) supplemented with 0.1% epinehrin (Biofluids/Biosource, catalog #355-002), 3.3 mM retinoic acid (Biofluids/Biosource, catalog #348-001) and penicillin-streptomycin-fungizone (Invitogen, catalog #15240-062) and were a gift from Drs. Suzy Comhair and Serpil Erzurum (Cleveland Clinic).

Construction of Cells with RNase L, PDE12, and AKAP7 Knockout.

The A549 RNase L KO cells were described previously [31]. Additional knockout cells were generated using the CRISPR Cas9 system. The sgRNA sequences were chosen from a published database [17]. The guide RNA sequences were synthesized as DNA oligonucleotides by IDT. Primers were annealed, phosphorylated and ligated into vector LentiCRISPR v2 (Addgene 52961) that was prepared by digestion with BsmBI. The resulting plasmids were transformed into Stbl3 chemically competent cells (Invitrogen) and grown on a bacterial culture plate at 37° C. Colonies were screened by PCR using the U6 primer and gene specific reverse primer to generate approximately 300 bp product. Positive clones were cultured and plasmid DNA was prepared and sequence verified using the U6 primer. The AKAP7 sgRNA sequences are: sgAKAP7-2 FW CACCG TGAGC GACTG GCCAA AGCAA (SEQ ID NO:1) and sgAKAP7-2 REV AAACT TGCTT TGGCC AGTCG CTCA C (SEQ ID NO:2). The PDE12 sgRNA sequences are: sgPDE12_10.FW CACCG GGATG CCTGG CAAGA CGGCG (SEQ ID NO:3) and sgPDE12_10.REV AAACC GCCGT CTTGC CAGGC ATCCC (SEQ ID NO:4). Cell cloning was done by limited dilution. The CRISPR vector, pLenti-sgRL-6, was used to knockout RNase L in PC3 cells as we described for RNase L KO A549 cells [17, 31]. To obtain the PDE12/AKAP7 DKO cells, a PDE12 knockout A549 cell line was infected with pseudo lentiviruses expression sgRNA for AKAP7 followed by single cell cloning. The PDE12/RNase L DKO cells were obtained by infecting the RNase L KO A549 cells with the pseudo lentivirus expressing sgRNA for AKAP7 followed by single cell cloning. Resulting clones were screened for knock-out of expression using Western Blots with anti-PDE12 antibody (Abcam ab87738), anti-AKAP7 (Proteintech: Cat#12591-1-AP) antibody or monoclonal antibody against human RNase L [13].

### Immunoblotting.

Prior to lysis, cells were washed twice in cold phosphate-buffered saline (PBS). Cell extracts were prepared with lysis buffer supplemented with phosphatase/protease inhibitors followed by incubation on ice for 20 min. Lysates were subjected to centrifugation at 12,000 g for 10 min, and the supernatants collected and protein quantified by Bradford assays (BioRaD). Cell lysates (30-50 pg) were separated on 8% or 10% SDS PAGE gels and proteins were transferred to polyvinylidene difluoride membranes (0.45 μm) (BioRaD) and probed with antibodies according to the different manufacturers' recommendations.

### Cell Death Assay.

Cells (20,000 per well) were seeded in 24-well plates or 8,000 cells per well in 96-well plates and treated as stated in figure legends. The cells were incubated with 250 nM Sytox-Green dye (Thermo Fisher), a nucleic acid stain that is an indicator of dead cells and which is impermeant to live cells, and 250 nM of cell permeable dye Syto™ 60-Red (ThermoFisher), which allows quantification of the total number of cells present in each field, using an IncuCyte Live-Cell Imaging System and software (Essen Instruments 2015A) for 72 hours. Cell death was measured by counting the green objects per/well (dead cells, green) and then normalizing to the total number of cells per/well (red objects) at each time point using IncuCyte software.

### Apoptosis Assay (Caspase 3/7).

Cells were seeded in 96-well plates and treated with Aza as above. The cells were incubated with IncuCyte™ Kinetic Caspase-3/7 Apoptosis Assay Reagent (green) according to manufacturer's protocol and with 250 nM of Syto Red dye (ThermoFisher) for 24 hours. The apoptotic index was determined by determining the total count [green Integrated Intensity (GCU×μm²/Image)] and then normalizing by count confluence (percent) using IncuCyte software.

### Results

### Deletion of RNase L Renders A549 Lung Cancer Cells Resistant to the Cell-Lethal Effect of AZA.

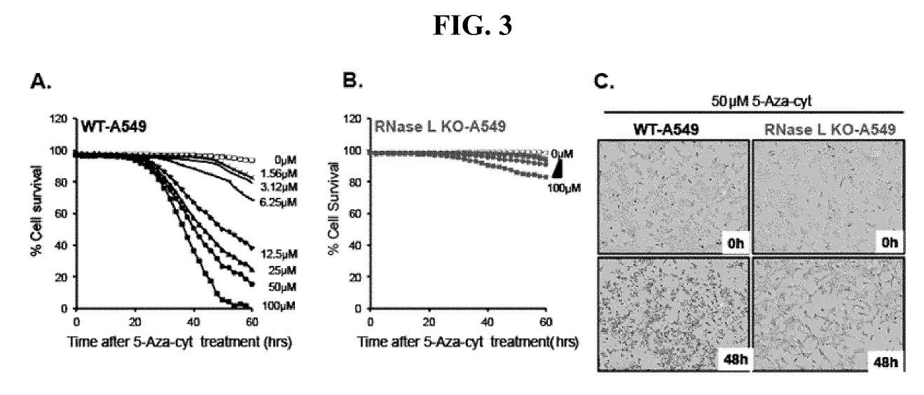

To determine the impact of RNase L on sensitivity to AZA of A549 non-small cell lung carcinoma cells, we compared the parental vector control A549 cells (referred to as wild type or WT) with RNase L KO cells, the latter generated with CRISPR-Cas9 technology [17]. The cells treated with different doses of AZA were monitored as a function of time for dead and total (live+dead) cells with a dual dye method in an IncuCyte real-time cell imaging system. The WT cells were highly sensitive (85% and 1000/% cell death) to 50 and 100 uM AZA, respectively, by 60 hours of treatment (FIG. 3A,C). In contrast, the RNase L KO cells were highly resistant with only 9% and 17% cell death in response to 50 and 100 uM AZA (FIG. 3B,C). While the present invention is not limited to any particular mechanism, and an understanding of the mechanism is not necessary to practice the invention, these results suggest that RNase L is required for the death of A549 lung cancer cells in response to AZA. The A549 are sensitive to AZA despite being wild type for p53 status (source: ATCC).

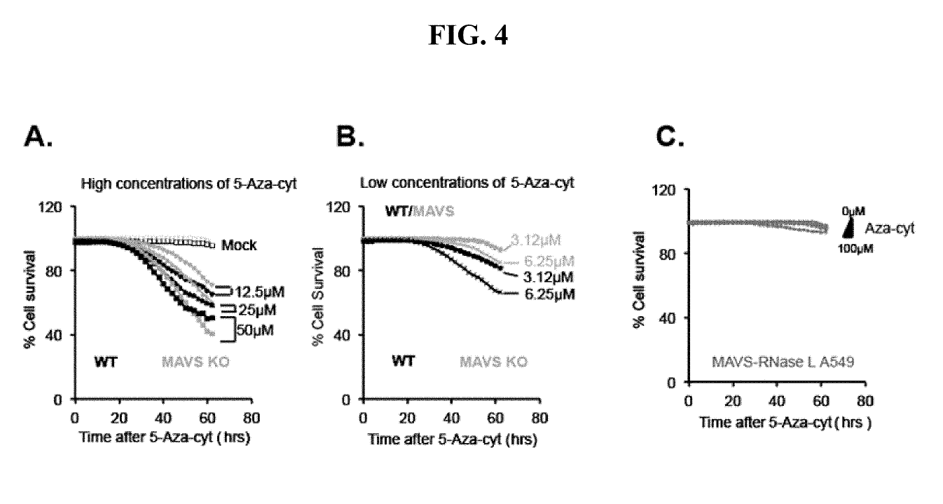

DsRNA signaling to the type I IFN genes requires the RIG-I-like (RLR) helicase/MAVS pathway[18]. Therefore, to determine if IFN signaling was required for the cell killing effect of AZA, MAVS was knocked out singly and in combination with RNase L (double knockout or DKO). MAVS KO A549 cells were slightly less sensitive to AZA treatment, suggesting that IFN production contributed to, but was not absolutely required for AZA-mediated cell death (FIG. 4A,B). As expected, however, the MAVS-RNase L DKO cells were almost completely resistant to AZA treatment as concentrations up to 100 uM (FIG. 4C). Results suggest that basal levels of OASs (products of IFN stimulated genes) are sufficient for the cell-lethal effect of AZA through RNase L activation.

### AZA Causes Apoptotic Death of A549 Cells.

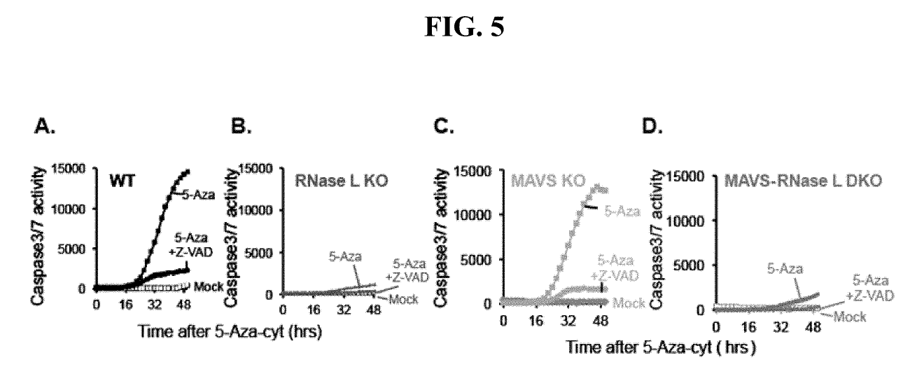

To determine the mode of cell death by AZA, caspase3/7 activation was monitored as a marker of apoptosis by real-time imaging with an IncuCyte System and software (FIG. 5). While WT A549 cells produce high levels of caspase3/7 activation in response to AZA, the RNase L KO A549 cells were resistant (FIG. 5A,B). MAVS KO had at best only a small effect in reducing caspase3/7 activation, whereas MAVS/RNase L DKO cells were highly resistant, similar to the single KO of RNase L (FIG. 5C,D). As a control, the pan-caspase inhibitor, Z-VAD, greatly suppressed caspase3/7 activation as expected.

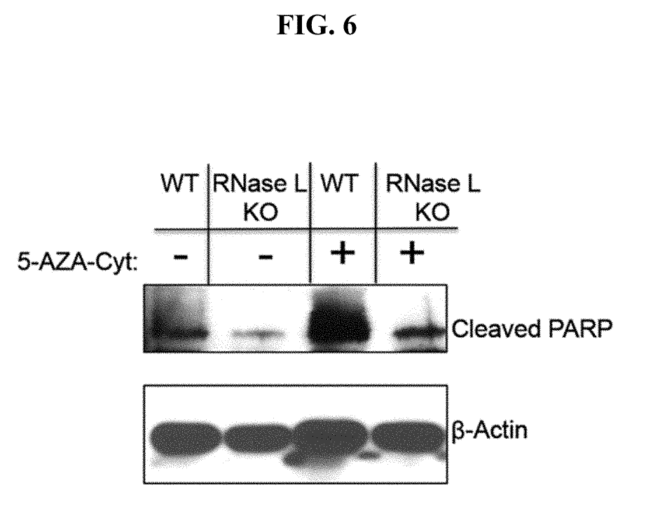

To confirm that AZA induced apoptosis, cleavage of PARP was monitored in Western blot assays. Treatment of WT A549 cells with 50 uM AZA for 48 hours resulted in a large increase in cleaved PARP (FIG. 6). In contrast, identical treatment of RNase L KO A549 cells caused only a slight increase in PARP cleavage. Thus both caspase3/7 activation and PARP cleavage studies suggest that AZA-mediated cell death through RNase L activation occurred by apoptosis.

### Primary Human Lung Epithelia are Resistant to AZA Treatment.

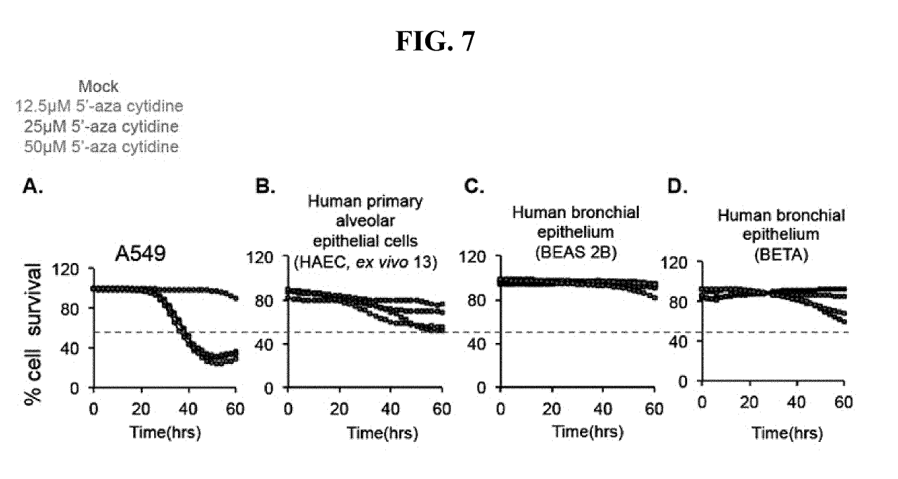

To determine compare the AZA sensitivity of A549 cancer cells to normal human lung epithelia, three cell types were examined, primary alveolar epithelial cells HAEC, bronchial epithelium BEAS 2B and BETA. Whereas control WT A549 were highly sensitive to AZA, the primary cells were resistant (FIG. 7).

### Sensitivity of Prostate Cancer Cells to AZA Treatment.

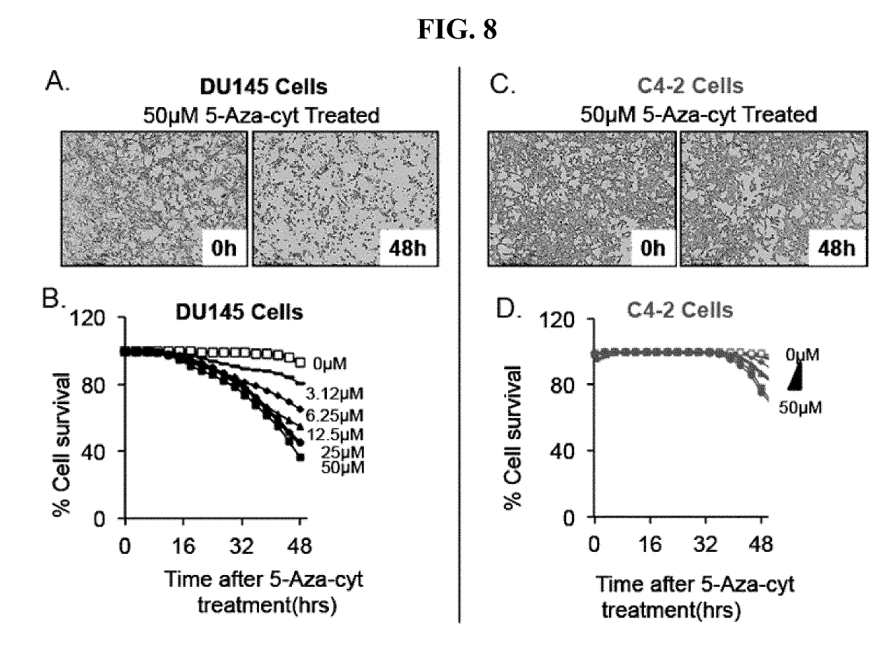

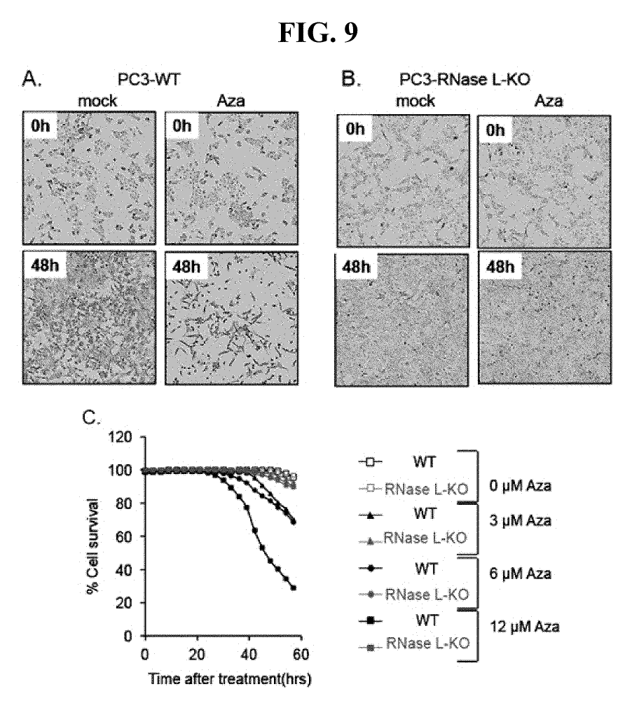

To extend these studies to another human cancer type, three prostate cancer cell lines were examined for the sensitivity to AZA (FIGS. 8, 9). DU145 and PC3 cells were sensitive to induction of cell death by AZA, whereas C4-2 were relatively resistant. C4-2 cells are derived from LNCaP which have a reduced activity variant of RNase L (R462Q) and a truncating mutation (E265X) in an RNase L allele [19]. Therefore, it is likely that low levels of RNase L activity in C4-2 cells result in reduced sensitivity to AZA.

### 2-5A Enhances the Sensitivity of A549 Cells to the Tumor Cell Killing Activity of AZA.

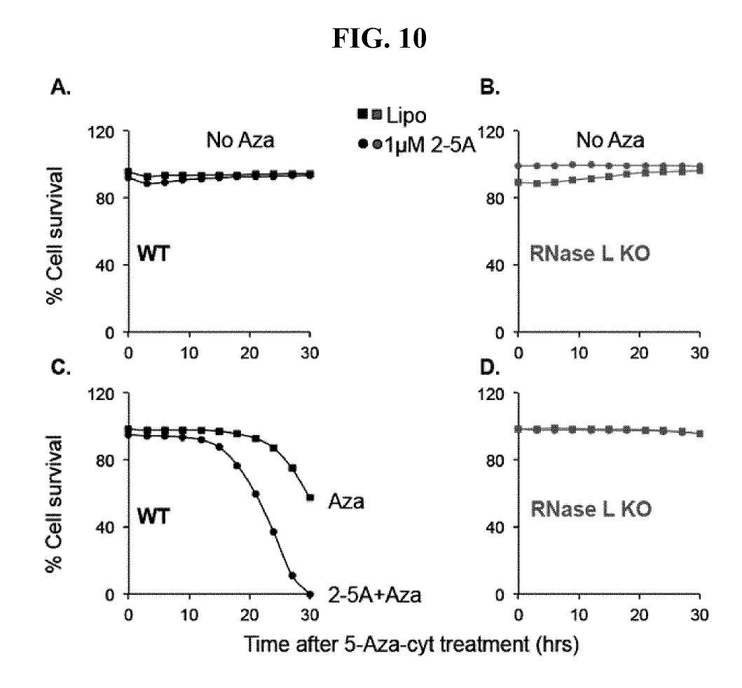

To determine if direct activation of RNase L would impact tumor cell killing by AZA, WT and RNase L KO A549 cells were treated with AZA, 2-5A or both agents (FIG. 10). Whereas a low dose of 2-5A (1 uM) transfection by itself had no effect on cell death or survival, and AZA (25 uM) by itself reduced WT A549 cell survival to 57%, the combination of 2-5A (1 uM) and AZA (25 uM) eliminated all live cells by 30 hours of treatment (FIG. 10A,C). In contrast, the RNase L KO A549 cells were completely resistant to AZA, 2-5A and the combination of both agents (FIG. 10B,D). These results demonstrate that 2-5A increases the cell-lethal effect of AZA and that the mechanism of action of the combination treatment of AZA and 2-5A is entirely dependent on RNase L.

Mutation of Host PDEs that Degrade 2-5A Enhance Tumor Cell-Killing by AZA Treatment.

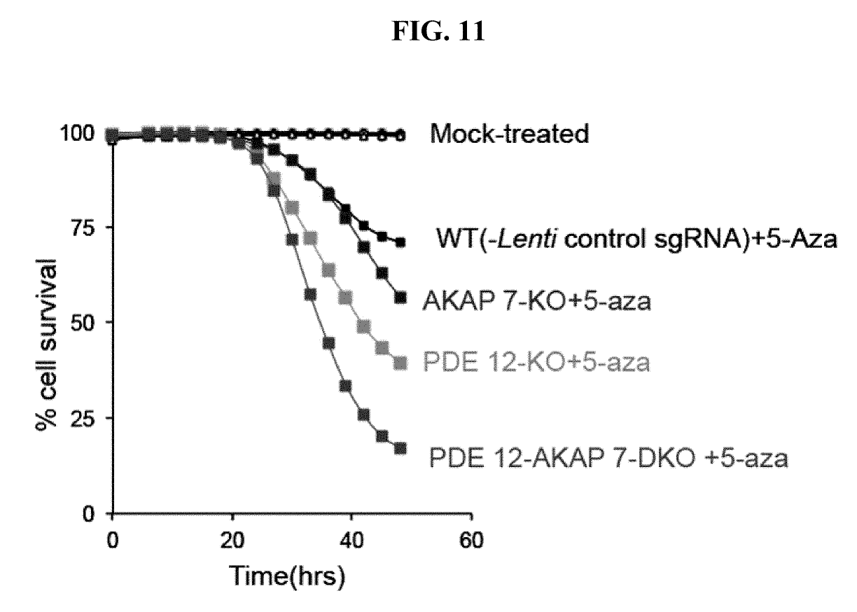

2-5A is degraded in cells through the action of the 2′,5′-phosphodiesterases, AKAP7 and PDE12 [20-22]. Therefore, to enhance 2-5A accumulation and RNase L activity during AZA treatments, the genes encoding AKAP7 and PDE12 were knocked out in A549 cells singly and in combination with CRISPR-Cas9 gene editing methods (FIG. 11). Deletion of AKAP7 moderately enhanced levels of cell death in the presence of AZA (56% cell survival), whereas PDE12 knockout had a larger effect (37% cell survival). The double knockout (DKO) for both AKAP7 and PDE12 greatly increased cell death from AZA treatment (17% cell survival). These results indicate that PDE inhibitors enhance the tumor cell-killing effect of AZA.

### The Tumor Cell-Lethal Effect of 5-Aza-2′-Deoxycytidine (DAC) is Dependent on RNase L.

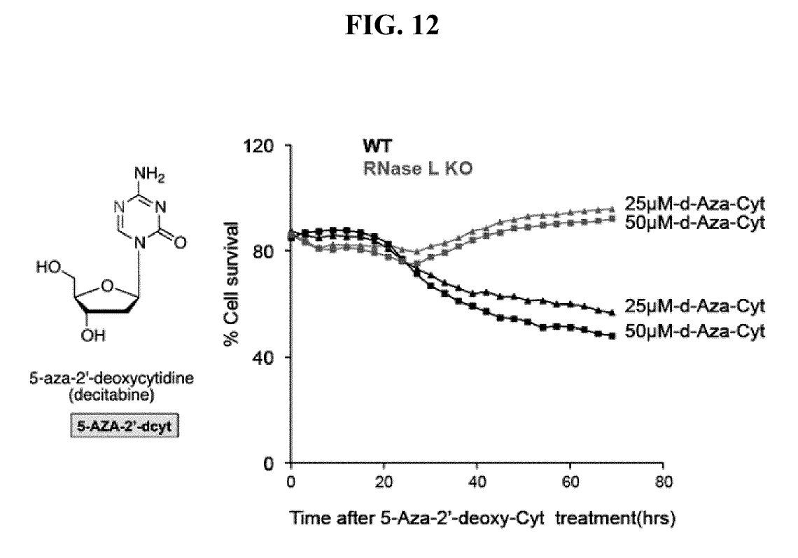

To extend these findings to 5-aza-2′-deoxycytidine (DAC), WT and RNase L KO A549 cells were treated with DAC for up to 70 hrs (FIG. 12). While WT A549 we less sensitive to DAC than to AZA, there was only 56% and 47% cell survival after treatment with 25 uM and 50 uM DAC. In contrast, there was no effect of DAC on the survival of RNase L KO A549. These finding suggest that either AZA or DAC could be used in combination with a 2′-PDE inhibitor or 2-5A to enhance the death of tumor cells.

DNMTi's have been investigated as potential cancer therapeutic agents for 45 years [23] and, currently, the DNA demethylating agent AZA is an FDA approved drug for the treatment of MDS [2-5]. Over the past 12 years, there have been several studies reporting links between of DNMT inhibitors effects on cancer cells and the IFN system. For example, DAC induced the expression of IFN stimulated genes (ISGs) in human pancreatic cancer cells [24] and another study showed that either DAC or antisense against DNA methyltransferase I (DNMT1) when used together with IFN-α2 or IFN-β promoted apoptosis of renal cell carcinoma ACHN or melanoma A375 cells, but not of normal kidney epithelial cells [25]. Small cell lung cancer (SCLC) cells that were resistant to combination of DNA methyltransferase and histone deacetylase inhibitors had higher basal expression of IFN-stimulated genes than sensitive SCLC cells[26]. Also, IFN-α enhanced the direct and immune mediated effects of AZA with histone deacetylase inhibitor romidepsin against colorectal cancer metastatic and stem cells [27]. Interestingly, the ability of DNMTi's to suppress tumor cell survival is linked to the production of self-dsRNA that triggers an IFN innate immune response, referred to as viral mimicry. AZA and DAC induced in cells with mutant p53 transcription of short interspersed elements (SINE), DNA encoding long non-coding RNAs (ncRNA), satellite DNA and ERV elements all of which result in dsRNA and an IFN stimulated transcriptional response [7-9]. It is noted however that the cancer cells used in this study are wild type for p53 and therefore dsRNA induction by AZA or and DAC are not limited to tumor cells with mutant p53. Also, vitamin C, a co-factor for ten-eleven translocation (TET) enzymes that oxidize 5-methyl cytosine, enhanced viral mimicry by expression of ERV cytosolic dsRNA in response to DAC [28].

OAS genes are ISGs that encode sensors of dsRNA [29, 30]. Upon binding dsRNA, OAS1-3 use ATP as substrate to synthesize unusual 2′,5′-linked oligoadenylates collectively referred to as 2-5A. The only well established function of 2-5A is activation of RNase L. RNase L is an antiviral protein with pro-apoptotic activity [12, 16]. The apoptotic effects of AZA or DAC are largely the result of the dsRNA-activated and IFN inducible OAS-RNase L pathway. We present here data on lung and prostate cancer cell lines in which AZA treatment leads to RNase L-induced cell death. Prior studies on anti-tumor activities of AZA and DAC showed effects against melanoma through enhanced immune checkpoint therapy [9] and against colorectal cancer through targeting of colorectal cancer-initiating cells [8]. An earlier study showed that DAC could overcome resistance to IFN-induced apoptosis in renal carcinoma and melanoma[25]. In that report the IFN effect was linked to the apoptosis-induced IFN response gene XAF1. However, because OAS genes are IFN inducible it seems likely based on our study that OAS-RNase L is primarily responsible for apoptosis and not XAF1.

Our findings indicate, for example, strategies for enhancing the anti-cancer effects of IFN. Combining AZA or DAC (or other DNA methylation inhibitor) with 2-5A (an RNase L enhancer) was highly effective in eliminating tumor cells. We also showed that knockout of PDE12 or AKAP7, which impair 2-5A degradation, enhanced tumor cell killing by AZA. Therefore, drugs that either increase RNase L activity directly, or that block PDE12 [21, 22] or AKAP7 increasing RNase L activity indirectly, will enhance the anti-tumor activity of AZA or DAC. Normal cell types appear to be relatively resistant to AZA, therefore such combination experimental therapies might selectively target cancer cells (FIG. 7).

### REFERENCES

1. Sampath et al., Mechanisms of apoptosis induction by nucleoside analogs. Oncogene. 2003; 22(56):9063-9074.
2. Hamm C A and Costa F F. Epigenomes as therapeutic targets. Pharmacol Ther. 2015; 151:72-86.
3. Nazha et al., and Consortium MDSCR. Outcomes of patients with myelodysplastic syndromes who achieve stable disease after treatment with hypomethylating agents. Leuk Res. 2016; 41:43-47.
4. Dawson M A. The cancer epigenome: Concepts, challenges, and therapeutic opportunities. Science. 2017; 355(6330): 1147-1152.
5. Dombret et al. International phase 3 study of azacitidine vs conventional care regimens in older patients with newly diagnosed AML with >30% blasts. Blood. 2015; 126(3):291-299.
6. Qiu et al., Aerosol azacytidine inhibits orthotopic lung cancers in mice through Its DNA demethylation and gene reactivation effects. PLoS One. 2014; 9(10):e109874.
7. Leonova et al., p53 cooperates with DNA methylation and a suicidal interferon response to maintain epigenetic silencing of repeats and noncoding RNAs. Proc Natl Acad Sci USA. 2013; 110(1):E89-98.
8. Roulois et al., DNA-Demethylating Agents Target Colorectal Cancer Cells by Inducing Viral Mimicry by Endogenous Transcripts. Cell. 2015; 162(5):961-973.
9. Chiappinelli et al. Inhibiting DNA Methylation Causes an Interferon Response in Cancer via dsRNA Including Endogenous Retroviruses. Cell. 2015; 162(5):974-986.
10. Silverman R H and Weiss S R. Viral phosphodiesterases that antagonize double-stranded RNA signaling to RNase L by degrading 2-5A. J Interferon Cytokine Res. 2014; 34(6):455-463.
11. Silverman R H. Viral encounters with 2′,5′-oligoadenylate synthetase and RNase L during the interferon antiviral response. J Virol. 2007; 81(23):12720-12729.
12. Zhou et al., Expression cloning of 2-5A-dependent RNAase: a uniquely regulated mediator of interferon action. Cell. 1993; 72(5):753-765.
13. Dong B and Silverman R H. 2-5A-dependent RNase molecules dimerize during activation by 2-5A. J Biol Chem. 1995; 270(8):4133-4137.
14. Huang et al., Dimeric structure of pseudokinase RNase L bound to 2-5A reveals a basis for interferon induced antiviral activity. Molecular Cell. 2014; In Press.
15. Zhou et al., Interferon action and apoptosis are defective in mice devoid of 2′,5′-oligoadenylate-dependent RNase L. Embo J. 1997; 16(21):6355-6363.
16. Castelli et al., A study of the interferon antiviral mechanism: apoptosis activation by the 2-5A system. J Exp Med. 1997; 186(6):967-972.
17. Wang et al., Genetic screens in human cells using the CRISPR-Cas9 system. Science. 2014; 343(6166):80-84.
18. Akira et al., Pathogen recognition and innate immunity. Cell. 2006; 124(4):783-801.
19. Xiang et al., Effects of RNase L mutations associated with prostate cancer on apoptosis induced by 2′,5′-oligoadenylates. Cancer Res. 2003; 63(20):6795-6801.
20. Gusho et al, Murine AKAP7 has a 2′,5′-phosphodiesterase domain that can complement an inactive murine coronavirus ns2 gene. MBio. 2014; 5(4):e01312-01314.
21. Wood et al. The Role of Phosphodiesterase 12 (PDE12) as a Negative Regulator of the Innate Immune Response and the Discovery of Antiviral Inhibitors. J Biol Chem. 2015. 290, 19681-19696.
22. Kubota et al., Identification of 2′-phosphodiesterase, which plays a role in the 2-5A system regulated by interferon. J Biol Chem. 2004; 279(36):37832-37841.
23. Weiss et al., Phase I study of 5-azacytidine (NSC-102816). Cancer Chemother Rep. 1972; 56(3):413-419.
24. Missiaglia et al., Growth delay of human pancreatic cancer cells by methylase inhibitor 5-aza-2′-deoxycytidine treatment is associated with activation of the interferon signalling pathway. Oncogene. 2005; 24(1):199-211.
25. Reu et al., Overcoming resistance to interferon-induced apoptosis of renal carcinoma and melanoma cells by DNA demethylation. J Clin Oncol. 2006; 24(23):3771-3779.
26. Luszczek et al., Combinations of DNA methyltransferase and histone deacetylase inhibitors induce DNA damage in small cell lung cancer cells: correlation of resistance with IFN-stimulated gene expression. Mol Cancer Ther. 2010; 9(8):2309-2321.
27. Buoncervello et al. IFN-alpha potentiates the direct and immune-mediated antitumor effects of epigenetic drugs on both metastatic and stem cells of colorectal cancer. Oncotarget. 2016; 7(18):26361-26373.
28. Liu et al., Vitamin C increases viral mimicry induced by 5-aza-2′-deoxycytidine. Proc Natl Acad Sci USA. 2016; 113(37):10238-10244.
29. Hovanessian et al., Synthesis of low molecular weight inhibitor of protein synthesis with enzyme from interferon-treated cells. Nature. 1977; 268(5620):537-540.
30. Kristiansen et al., The oligoadenylate synthetase family: an ancient protein family with multiple antiviral activities. J Interferon Cytokine Res. 2011; 31(1):41-47.
31. Li et al., Activation of RNase L is dependent on OAS3 expression during infection with diverse human viruses. Proc Natl Acad Sci USA. 2016; 113(8):2241-2246.

### Example 2

This Example describes additional experimental work that was conducted.

### Cells Lacking RNase L or OAS1-3 are Highly Resistant to the Cell-Lethal Effect of AZA.

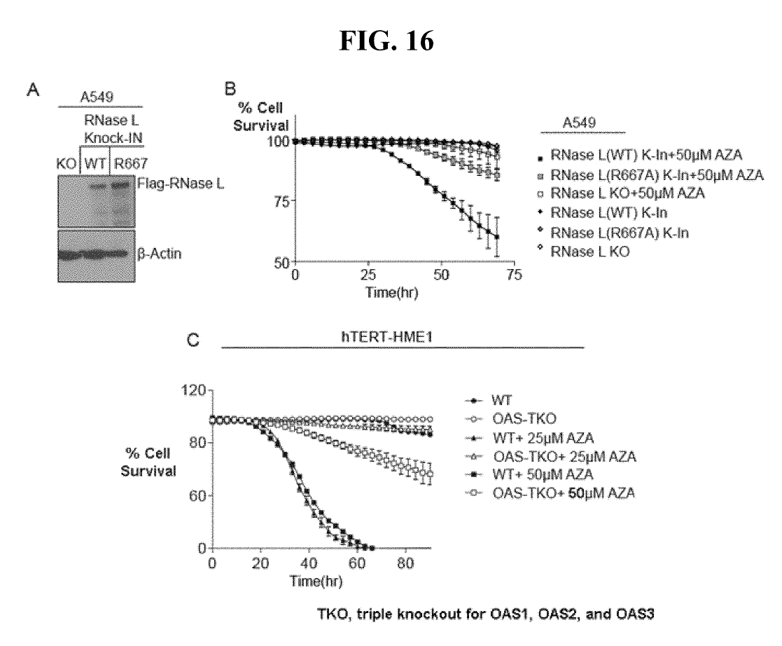

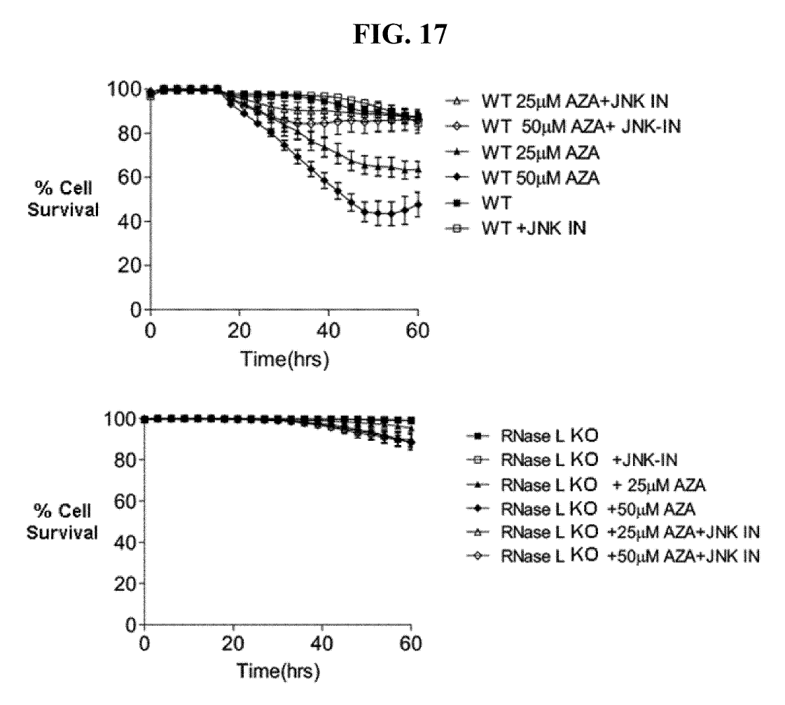

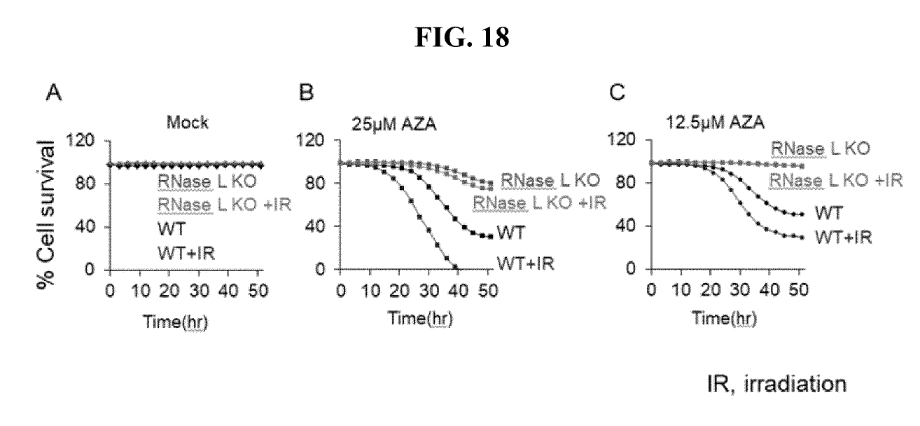

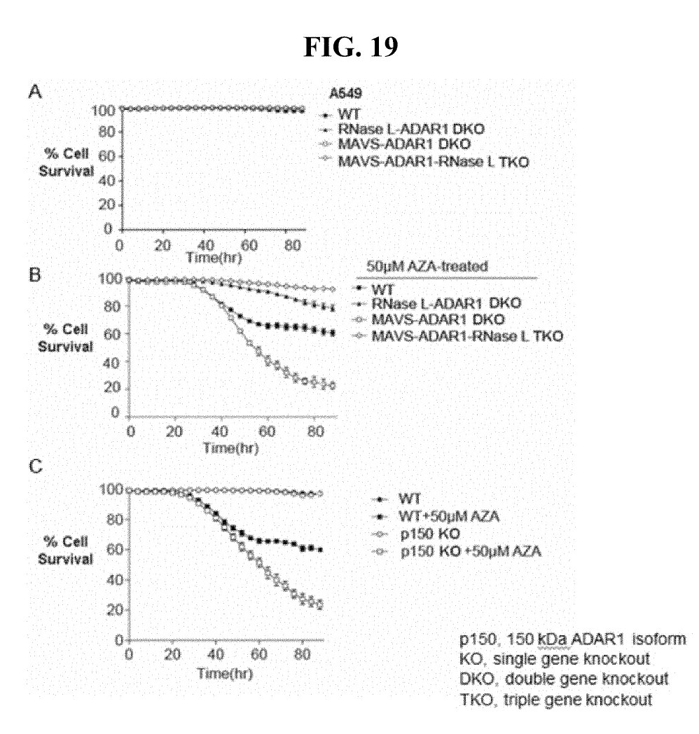

To further confirm that RNase L was responsible for AZA sensitivity, RNase L KO A549 cells were transiently transduced with lentiviruses encoding either wild type (WT) or nuclease-dead mutant (R667A) RNase L (1) (FIG. 164). Knock-in (K-in) of WT RNase L sensitized cells to AZA treatment, whereas the knock-in of mutant RNase L (R667A) did not (FIG. 16B).

As an alternative to RNase L knockout, the three catalytically active OAS isoforms (OAS1, 2&3) were knocked out by means of CRISPR-cas9 gene editing methods generating triple knockout (TKO) cells (FIG. 16C). Immortalized human mammary epithelial cells (hTERT-HME1) were used. The absence of OAS1-3 rendered the HME cells relatively resistant to AZA, supporting the central role of the OAS-RNase L pathway in mediating cell death in response to AZA.

A Small Molecule Inhibitor of c-Jun NH2-Terminal Kinase (JNK) Prevents Cell Death by AZA in an RNase L-Dependent Pathway.

The c-Jun NH2-terminal kinase (JNK) family of MAP kinases relays signals from a wide range of extracellular stimuli including viruses, cytokines, and environmental stress. JNKs often play a critical role in controlling the balance between cell survival and death. Previously, we reported that JNK and RNase L function in an integrated signaling pathway during the IFN response that leads to elimination of virus-infected cells through apoptosis(2). RNase L causes apoptosis through a ribotoxic-stress pathway involving JNK signaling(2). The anthrapyrazolone inhibitor, SP600125, is a reversible, ATP-competitive inhibitor for JNKs and other several other kinases(3). Consistent with the involvement of RNase L in AZA-induced cell death, we observed that the JNK inhibitor SP600125 suppressed apoptosis by AZA treatment (FIG. 17).

### The Cytotoxic Response of Ionizing Radiation and AZA is Dependent Upon RNase L.

Previously, AZA induced cytotoxicity was linked to reversible DNA damage stemming from covalent complexes between DNA and DNMTs (4, 5). Also, during DNA damage from ionizing irradiation or chemotherapy agents, U1 and U2 small non-coding RNAs accumulate in the cytoplasm activating the pathogen recognition receptor RIG-I and IFN signaling(6). Those results suggested that DNA damage from AZA might further induce dsRNA activators of OAS. In support of this idea, we show that ionizing radiation (IR) potentiates the cytotoxicity of AZA through RNase L (FIG. 18). Interestingly, many different primary malignant tumors, including head and neck, lung, prostate, breast, and high-grade glioma, express high levels of OAS proteins as part of the IFN-related DNA damage resistance signature (IRDS)(7). Therefore, AZA-induced cell death through OAS-RNase L might preferentially target cancer cells in vivo during IR therapy of cancer.

### ADAR1 Knockout Increases the Susceptibility of Cells to AZA.

ADAR1 (double-stranded RNA-specific adenosine deaminase) destabilizes dsRNA by adenosine-to-inosine RNA editing (8-10). Genetic deficiency in ADAR1 leads to RNase L-dependent cell death (11) and IFN-stimulated translational arrest through PKR(12). There are similarities between cellular effects of AZA treatment and ADAR1 mutation in that both lead to self dsRNA accumulation transcribed from repetitive DNA elements, including Alu (SINE) and hERV elements (12-14). In the case of ADAR1 mutation, it is the deficiency in self dsRNA editing that leads to enhanced activation of OAS-RNase L, whereas during AZA treatment it is enhanced transcription of repetitive DNA elements that activates OAS-RNase L. ADAR1 deletion is cell-lethal, therefore we used MAVS-ADAR1 DKO cells in the experiments (15).

To determine if ADAR1 antagonizes the cell-lethal effect of AZA, A549 cells of different ADAR1, MAVS and RNase L genotypes were treated with AZA. Cell lacking RNase L alone (RNase L KO), triple knockout (TKO) of MAVS-ADAR1-RNase L and double knockout (DKO) of ADAR-RNase L were AZA resistant (FIG. 19A,B). In contrast, MAVS-ADAR1 DKO cells, which express RNase L, showed enhanced AZA sensitivity compared with WT cells, presumably from increased accumulation of self dsRNA due to absence of ADAR1 (FIG. 19B). Similarly, ADAR1 p150 KO (the IFN inducible ADAR1 isoform) was highly sensitive to the cytotoxic effect of AZA (FIG. 19C). These results indicate that AZA sensitivity of cells can be increased by preventing ADAR1 RNA editing. This could be accomplished by means of an ADAR1 inhibitor drug (e.g., 8-azaadenosine).

### REFERENCES (FOR EXAMPLE 2 ONLY)

1. Dong et al., (2001) Basis for regulated RNA cleavage by functional analysis of RNase L and Ire1p. Rna 7(3):361-373.
2. Li et al., (2004) An apoptotic signaling pathway in the interferon antiviral response mediated by RNase L and c-Jun NH2-terminal kinase. J Biol Chem 279(2):1123-1131.
3. Bennett, et al. (2001) SP600125, an anthrapyrazolone inhibitor of Jun N-terminal kinase. Proc Natl Acad Sci USA 98(24):13681-13686.
4. Palii et al., (2008) DNA methylation inhibitor 5-Aza-2′-deoxycytidine induces reversible genome-wide DNA damage that is distinctly influenced by DNA methyltransferases 1 and 3B. Mol Cell Biol 28(2):752-771.
5. Kiziltepe, et al. (2007) 5-Azacytidine, a DNA methyltransferase inhibitor, induces ATR-mediated DNA double-strand break responses, apoptosis, and synergistic cytotoxicity with doxorubicin and bortezomib against multiple myeloma cells. Mol Cancer Ther 6(6):1718-1727.
6. Ranoa, et al. (2016) Cancer therapies activate RIG-I-like receptor pathway through endogenous non-coding RNAs. Oncotarget 7(18):26496-26515.
7. Weichselbaum, et al. (2008) An interferon-related gene signature for DNA damage resistance is a predictive marker for chemotherapy and radiation for breast cancer. Proc Natl Acad Sci USA 105(47):18490-18495.
8. George et al., (2014) An RNA editor, adenosine deaminase acting on double-stranded RNA (ADAR1). J Interferon Cytokine Res 34(6):437-446.
9. Tomaselli et al., (2015) ADARs and the Balance Game between Virus Infection and Innate Immune Cell Response. Curr Issues Mol Biol 17:37-51.
10. Patterson J B & Samuel C E (1995) Expression and regulation by interferon of a double-stranded-RNA-specific adenosine deaminase from human cells: evidence for two forms of the deaminase. Mol Cell Biol 15(10):5376-5388.
11. Li, et al. (2017) Ribonuclease L mediates the cell-lethal phenotype of the double-stranded RNA editing enzyme ADAR1 in a human cell line. Elife 6.
12. Chung, et al. (2018) Human ADAR1 Prevents Endogenous RNA from Triggering Translational Shutdown. Cell 172(4):811-824 e814.
13. Chiappinelli, et al. (2015) Inhibiting DNA Methylation Causes an Interferon Response in Cancer via dsRNA Including Endogenous Retroviruses. Cell 162(5):974-986.
14. Roulois, et al. (2015) DNA-Demethylating Agents Target Colorectal Cancer Cells by Inducing Viral Mimicry by Endogenous Transcripts. Cell 162(5):961-973.
15. Li, et al. (2008) An essential role for the antiviral endoribonuclease, RNase-L, in antibacterial immunity. Proc Natl Acad Sci USA 105(52):20816-20821.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of treating cancer comprising: administering to a subject with cancer a first agent and a second agent,
wherein said first agent comprises a PDE12 inhibiting agent, a AKAP7 inhibiting agent, an ADAR1 inhibiting agent, and/or a RNase L enhancer, and
wherein said second agent comprises a DNA methylation inhibiting agent.

2. The method of claim 1, wherein said DNA methylation inhibiting agent comprises a DNA methyltransferase demethylase inhibitor.

3. The method of claim 1, wherein said DNA methylation inhibiting agent comprises 5-azacytidine (AZA).

4. The method of claim 1, wherein said DNA methylation inhibiting agent comprises 5-aza-2′-deoxycytidine (DAC).

5. The method of claim 1, wherein said first agent and said second agent are part of the same composition.

6. The method of claim 1, wherein said first agent is in a first composition and said second agent is in a second composition.

7. The method of claim 1, wherein said first agent is said RNase L enhancer which comprises the 2-5A RNase L activator compound.

8. The method of claim 1, wherein said first agent is said PDE12 inhibiting agent which is selected from the group consisting of: an anti-PDE12 siRNA sequence, an anti-PDE12 antisense sequence, polyclonal anti-PDE12 antibodies, a small molecule inhibitor of PDE12, and an anti-PDE12 monoclonal antibody or antigen binding fragment thereof.

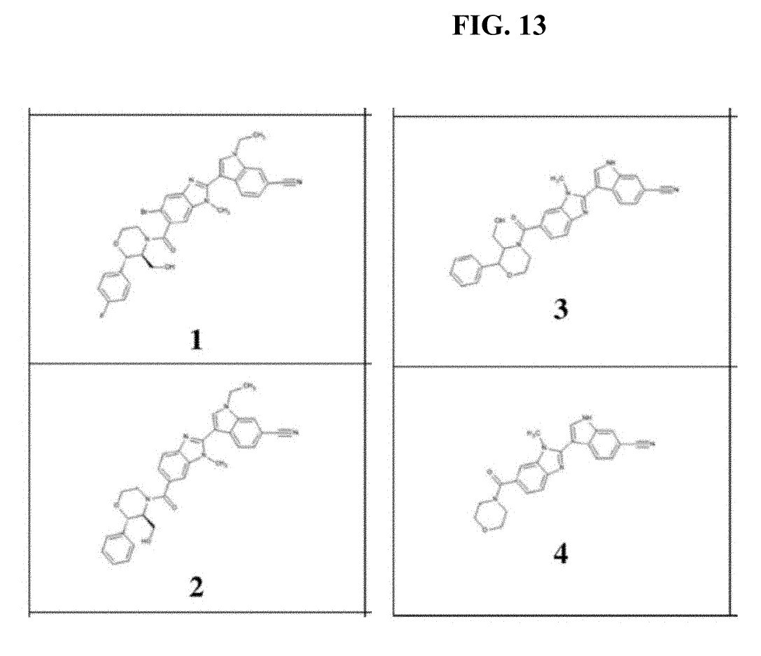

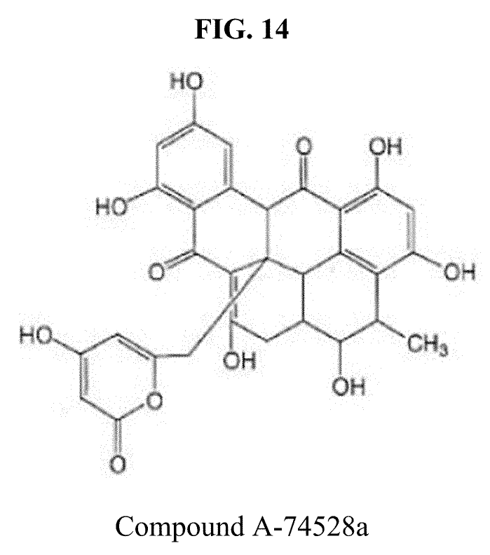

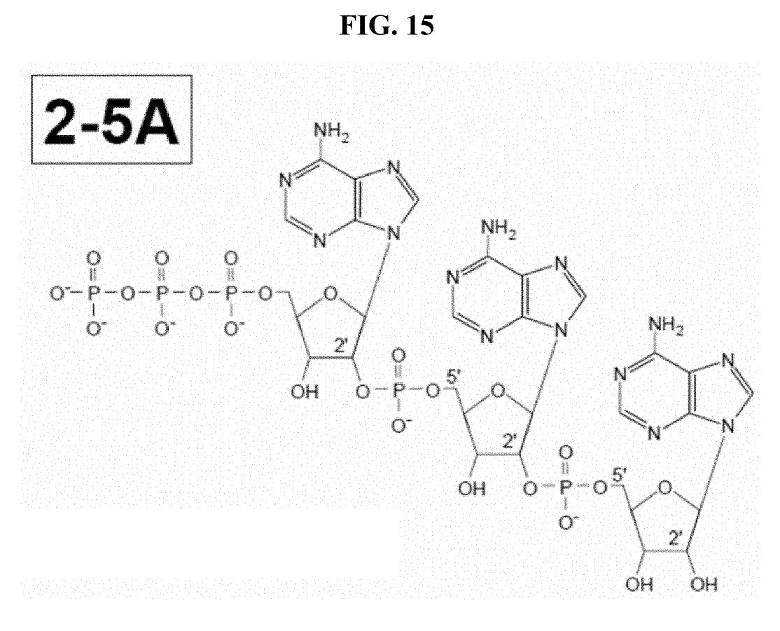

9. The method of claim 1, wherein said first agent is said PDE12 inhibiting agent is selected from the group consisting of: Compounds 1-4 shown in FIG. 13, or compound A-74528a FIG. 14.

10. The method of claim 1, wherein said first agent is said AKAP7 inhibiting agent is selected from the group consisting of: an anti-AKAP7 siRNA sequence, an anti-AKAP7 antisense sequence, anti-AKAP7 polyclonal antibodies, a small molecule inhibitor of AKAP7, or an anti-AKAP7 monoclonal antibody or antigen binding fragment thereof.

11. The method of claim 1, wherein said cancer comprises a solid malignant cancer, leukemia, or a myelodysplastic syndrome cancer.

12. The method of claim 1, wherein said administering reduces the level of cancer in said subject.

13. The method of claim 1, wherein said administering said first and second agents reduces the level of cancer in said subject by a larger amount than administering just said second agent.

14. The method of claim 1, wherein said subject is a human.

15. The method of claim 1, wherein said first agent is said ADAR1 inhibiting agent.

16. The method of claim 15, wherein said ADAR1 inhibiting agent comprises 8-azaadenosine.

17. The method of claim 1, further comprising treating said subject with ionizing radiation.

18. A system, kit, or composition comprising:
a) a first agent comprising a PDE12 inhibiting agent, a AKAP7 inhibiting agent, an ADAR1 inhibiting agent, and/or a RNase L enhancer, and
b) a second agent comprising a DNA methylation inhibiting agent.

19. The system, kit, or composition of claim 17, wherein said first and second agent are present in the same composition.

20. The system, kit, or composition of claim 17, wherein said first agent is present in a first container, and said second agent is present in a second container.


SEQUENCE LISTING (4 sequences)

SEQ ID NO: 1 - DNA, 25 bp, Artificial sequence
  Synthetic
caccgtgagcgactggccaaagcaa

SEQ ID NO: 2 - DNA, 25 bp, Artificial sequence
  Synthetic
aaacttgctttggccagtcgctcac

SEQ ID NO: 3 - DNA, 25 bp, Artificial sequence
  Synthetic
caccgggatgcctggcaagacggcg

SEQ ID NO: 4 - DNA, 25 bp, Artificial sequence
  Synthetic
aaaccgccgtcttgccaggcatccc